SEQUENCE LISTING (2 sequences)

SEQ ID NO: 1 - protein, 21 aa, synthetic construct
KGFYKKKQCRPSKGRKRGFCW

SEQ ID NO: 2 - protein, 21 aa, synthetic construct
KGFYKRKQCKPSRGRKRGICW

(19) United States
(12) Patent Application Publication
(10) Pub. No.: US 2026/0265319 A1
(43) Pub. Date: Sep. 10, 2026

(54) TRANSCRIPTIONAL CHOREOGRAPHY OF SKIN CELLS BY LIGANDED IMMODULIN PEPTIDES

(71) Applicant: Desmond Mascarenhas, Auburn, CA (US)

(72) Inventor: Desmond Mascarenhas, Auburn, CA (US)

(21) Appl. No.: 19/282,398

(22) Filed: Jul. 28, 2025

Related U.S. Application Data
(60) Provisional application No. 63/768,280, filed on Mar. 7, 2025.

(51) Int. Cl. C07K 14/47; A61K 8/64; A61K 8/73; A61K 8/86; A61K 9/00; A61K 9/50; A61K 38/00; A61Q 19/00; C07K 14/00

(52) U.S. Cl. CPC C07K 14/4702; A61K 8/64; A61K 8/735; A61K 8/86; A61K 9/0014; A61K 9/5031; A61K 9/5036; A61K 9/5052; A61Q 19/00; C07K 14/001; A61K 38/00; C07K 2319/00

(57) ABSTRACT

SCDIs mediate transcriptional effects through RXR transcriptional complexes. Biodistribution studies show that, when applied transdermally to rats using a nanoemulsion, SCDIs rapidly partition to skin tissue resulting in a 3-10× enrichment relative to plasma even at skin locations distant from the site of application. Transcriptional governance of circuits relevant to tissue repair, such as CD169+ macrophage differentiation, previously shown to drive differentiation of CD4+ Tcells into iTregs, can be increased by SCDIs with an RARα/β ligand; IL-19, IL-22 and IL-24 production by HaCaT keratinocytes with an RARγ agonist; and FGF7/KGF-1, TGFβ and COL1A1 by a partial PPARγ ligand in dermal fibroblasts. Myoblast or myofibroblast differentiation can be stimulated by a PPARα ligand. Sequential controlled-release of the appropriate SCDIs applied as a single dose, especially with transferrin as carrier, can drive transcriptional choreography in the context of a multi-stage biological process in tissue.

### TECHNICAL FIELD

This non-provisional patent application relates to a method for executing a novel technology termed “transcriptional choreography”, wherein a single agent administered to a mammalian tissue sets up a series of transcriptional programs for targeted, temporally precise modulation of multiple steps in a multi-stage biological process such as wound healing. The technology provides modified immodulin peptides that serve as versatile scaffolds for mechanistic, anatomical and temporal precision of action, leading to safer and more specific therapeutics or cosmetics employing transcriptional choreography. In light of their adjustable mechanistic specificity, side-chain-derivatized immodulins (SCDIs) are powerful new tools for transcriptional modulation, providing practitioners with an enhanced degree of anatomical precision, bioavailability and safety, when employing the compositions, methods and formulations herein disclosed. This breakthrough technology may have particular relevance in the burgeoning fields of distance practice and self-administered agents.

### RELATED APPLICATIONS AND PUBLICATIONS

The invention disclosed in this non-provisional patent application is related to content disclosed in U.S. application Ser. No. 18/407,753 which claims benefit of PCT/US2023/013908 with an international filing date of 26 Feb. 2023, which claims benefit of international application number PCT/US2022/018449 with an international filing date of 2 Mar. 2022, which claims benefit of international application number PCT/US2021/046814 with an international filing date of 20 Aug. 2021 and international application number PCT/US2021/021433 with an international filing date of 9 Mar. 2021. PCT/US2021/021433 claims priority to international application number PCT/US2020/024828 with an international filing date of 26 Mar. 2020. All of the above applications are hereby incorporated by reference into this specification. Unlike those applications, this invention provides specific methods for accomplishing multi-step transcriptional choreography in a single-dose format. Two recent publications (Mascarenhas et al, 2025; Mascarenhas 2022) are incorporated by reference into this specification. This non-provisional application claims benefit of provisional Appl. No. 63/768,280 filed on Mar. 7, 2025 entitled “TRANSCRIPTIONAL MODULATION OF SKIN CELLS BY LIGANDED IMMODULIN PEPTIDES”.

### BACKGROUND AND SUMMARY OF THE INVENTION

Transdermal delivery of controlled-release SCDIs can be used in the transcriptional choreography of skin cells (in vivo or ex vivo) to govern the sequential production of defined protein sets within the context of a multi-stage biological process. These compositions and methods are new in multiple ways.

Natural biological processes such as wound healing require a particular choreography of transcriptional events within the cells of living mammalian tissue. In healthy tissue, transcriptional events are triggered within tissues by environmental insult or even human intervention (such as deliberately wounding a tissue) thereby launching a multi-stage sequence of the natural healing processes. This type of sequential choreography underlies nature's ability to legislate specific and timely programming of desired events within each of the relevant cell types of a living tissue. In the skin of mammals, keratinocytes, fibroblasts and immune cells are important examples of cell types involved in this kind of epigenetic synchrony involving separate transcriptional events in tissue co-habiting cell types.

When a living tissue is unable or slowed by disease or age in the generation of desired transcriptional events in support of healing tissue, the desired process may become defective. For example, it is well known that diabetic wounds are notoriously slower (or even arrested) in healing, compared to normal tissue. It is therefore desirable for the medical and cosmetic industries to develop technologies that can rectify, enhance or accelerate the transcriptional choreography of such natural processes in order to produce healthier and/or better-looking skin.

To date, a technology that effectively achieves such precision in anatomical, mechanistic and temporal terms has remained a next-generation dream. Anatomically targeted, mechanistically precise and temporally coordinated transcriptional sequencing in a living mammalian tissue is exceedingly difficult to accomplish through the application of previously disclosed single-dose external agents using state-of-the-art agent design and delivery technologies. Part of the challenge is knowing which transcriptional events to influence, what tools can accomplish those tasks, and in what sequence those tools should be allowed to act. The current invention discloses, for the first time, how new agents and methods can accomplish these hitherto difficult or impossible feats both with exquisite precision and cost-effectiveness.

A recent publication (Mascarenhas et al, 2025) details a significant new tool for achieving precise transcriptional choreographies in human skin cells. Using that tool, a method for achieving precise sequencing of tissue healing steps is disclosed here. Most importantly, this invention enumerates specific nuclear receptor ligands to be used and in what order. For example, in one embodiment, an SCDI enhancing RARα/β action is followed by another SCDI enhancing PPARγ. In another embodiment, an SCDI enhancing RARα/β action is followed by the action of another SCDI enhancing RARγ. Knowing which tools to use in sequence is essential to achieving useful transcriptional choreography. The methods of the invention comprise contacting or administering specified side-chain-modified immodulin peptides (SCDIs) to mammalian skin cells or tissue, formulated so that they can work in a temporal order. Immodulin peptides (Mascarenhas, 2022) are unique peptide sequences derived from human proteins capable of rapidly entering living mammalian cells via an iron-transport pathway, entering the nucleus by an importin-mediated pathway, and then binding to a particular nuclear class of transcriptional complexes comprising certain isoforms of the master transcriptional factor retinoid X receptor (RXR). Various actions of immodulin peptides were described in that publication (Mascarenhas, 2022). A more recent publication (Mascarenhas et al, 2025) provides new insights in the use of SCDIs for achieving greater anatomical, mechanistic and temporal specificity.

The retinoid X receptor (RXR) is a type of nuclear receptor found ubiquitously in mammalian cells, whose actions are modulated by binding of dozens of known heterodimeric partners. RXR itself can be activated by 9-cis retinoic acid and numerous other ligands. Dozens of other ligands can activate or inhibit specific heterodimeric partners of RXR. Some of these ligands are natural, and some are designed. Depending on the nuclear concentrations of RXR heterodimeric partners in any given cell type, and subject to environmental cues, RXR dimers can direct appropriate transcriptional programs in response to challenges faced by living cells.

SCDIs are unique modifiers of RXR action. Their side-chain modifications are believed to act as bait for particular RXR heterodimers, thereby privileging the choice of transcriptional events in a cell's nuclear environment. Combined with timed-release formulations of SCDI peptide, this invention teaches the method of contacting mammalian tissue so as to accelerate a biological process in a stepwise fashion—an instantiation of which can be observed in accelerated natural healing, or in the resolution of inflammation in human skin.

One novel component of the invention is the anatomical targeting of a specific mammalian tissue such as skin. The above-referenced publication (Mascarenhas et al, 2025) shows that SCDIs applied externally to skin can concentrate in that tissue at about an order of magnitude higher concentration than in blood. The effect is not local i.e. SCDI applied to calf skin concentrates in that characteristically privileged manner at other skin sites too (arm, back), and at similarly high concentrations relative to plasma tissue. The safety implications of this natural anatomical targeting feature of SCDIs cannot be over-emphasized.

Another novel component of the invention is the mechanistic specificity and efficiency conferred by the transcriptional targeting made possible by using a particular SCDI side chain at very low dose. Relevant biological effects can be achieved at nanomolar concentrations in skin cells (Mascarenhas et al, 2025), several orders of magnitude lower than what would be required to see measurable effects by injection of free ligands into blood. Much higher circulating concentrations of free ligand (typically >10 μM at Cmax in known cases such as bexarotene, an approved drug) would need to be injected into the blood in order to achieve similar transcriptional outcomes, and it should be noted that in such modalities, all bodily tissues would be exposed to such high circulating doses, including tissues of critical visceral organs in which drug toxicities are often observed. The safety implications of mechanistic specificity, anatomical targeting and low-dose efficacy cannot be over-emphasized.

Yet another novel component of the invention is the use of recombinant transferrin protein in timed-release formulations. Transferrin binds immodulin peptides and is believed to act as a natural transporter for delivery of these peptides to ubiquitous cell surface receptors involved in iron metabolism. Timed release can be achieved by packaging SCDIs together with recombinant holo-transferrin in polymers that will release the peptide at a prescribed time, an encapsulation technique well-known in the art. Recovery of peptide from such packaged forms is not always efficient. The inclusion of the transporter transferrin helps maximize recovery of the peptide from polymer-encapsulated formulations (Mascarenhas et al, 2025) and also maximizes delivery of the peptide to cells in vivo once released. These practical features can dramatically minimize cost-of-goods and doses required (and thus, safety) as well, the overall holistic efficiency of the proposed processes cannot be over-emphasized.

A key novel element of the invention as disclosed here is the recitation of specific SCDI side chains in a particular order of delivery, wherein such transcriptional sequencing is designed to modulate a particular series of steps in a biological process in a mammalian tissue such as skin. The delivery of such transcriptional “ordering programs” at defined intervals, such as 12- or 24 hour intervals can be achieved by formulations known to those who are well-versed in the art. It is, however, necessary to know the proper sequencing and the desired spacing of transcriptional events in a tissue. This type of knowledge can be new and enabling. It is important to realize that immodulins have been described in scientific papers, patents and patent applications. Essentially all of this prior art has been generated by the inventor's work. What is new in this invention is the knowledge of the sequence of transcriptional events to be choreographed, which specific tools (such as ligands of nuclear receptors) can help achieve the desired result, and methods for using this critical new knowledge and the exquisite new tools (SCDIs) required, together with novel formulations containing recombinant transferrin. Methods and compositions herein disclosed are based on that new knowledge and are thus key enablers of embodiments of the invention. Such methods are critically useful in achieving the temporal specificity required for optimal impact on the specific biological process the operator wishes to influence. To summarize, the present invention teaches a level of anatomical, mechanistic and temporal specificity in transcriptional choreography unlike any hitherto achieved in a live mammalian tissue at nanomolar levels of applied agent. It also presages a whole new approach to drug and cosmetic development wherein safety concerns might be diminished by orders of magnitude.

Thus, the core components of the invention lie in the use of novel compositions and methods to affect transcriptional programs in skin tissue in the proper sequence (“transcriptional choreography”). The measurable outcomes are three-fold: temporal choreography of key transcripts, temporal choreography of key proteins specified by those transcripts, and the corresponding benefit in a multi-stage biological process such as the resolution of inflammation, or the healing of a wound. It is important to note that the invention goes beyond novel tools, to specifying which tools to deploy temporally (via controlled release formulations, for example), and in what precise order and spacing, in order to best benefit an underlying biological process. The sequential nature of the transcriptional solution is new. The knowledge disclosed regarding which key SCDIs, resulting transcripts and measurable proteins can be temporally monitored in skin tissue is also new.

Some notable elements of the invention include: (1) anatomical precision of delivery to a tissue e.g. skin; (2) efficiency and mechanistic precision of action by binding and modulating a specific transcriptional complex at nanomolar concentrations; (3) the ability to do so with multiple transcriptional ligands in the same cell at nanomolar concentrations, something that cannot be done in a practical way using free ligands; (4) temporal precision by determining the order of transcriptional sequencing (via selection of the appropriate SCDIs) and staggered delivery of said SCDIs. Staggered delivery can be achieved by providing each SCDI to the tissue in separate dosing events, or by formulating multiple SCDIs in a manner that leads to their orderly staggered release within desired timeframes; and (5) knowledge-based determination of the order/sequence of SCDIs whose sequential actions will, in aggregate, suitably synchronize with multi-stage natural processes in the tissue.

In one embodiment, this type of choreography can be described as the “use of (or method for achieving) a temporally staggered delivery of two distinct SCDIs (i.e. immodulin peptides whose side chains are covalently modified by different NR ligands) whose distinct transcriptional actions modulate temporally distinct steps in a natural multi-stage biological process involving crosstalk between signaling molecules of at least two different cell types in the same target tissue.” The two “different” or “distinct” SCDIs are peptides covalently attached to NR ligands. It is understood that a particular NR ligand modulates the action of its cognate NR, when delivered in sufficient dose to a cell nucleus containing the targeted transcriptional complex in sufficient dose.

Biomodulation of transcription in one cell type (e.g. an immune cell) may have cascading effects on other cell types. An SCDI that drives differentiation of CD169+ macrophages can help determine the types of T-cells generated in an environment. An SCDI that drives differentiation of keratinocytes can help determine the type of cytokine (e.g. IL-19) generated in an environment, wherein secondary effects of that cytokine (on fibroblasts, for example) are a secondary result. Thus the process may also be described as “temporally staggered delivery of two distinct SCDIs so as to transcriptionally modulate a multi-stage biological process involving a symphony of crosstalk between different cell types in the same target tissue”. Or, in a different form: “Temporally staggered delivery of two distinct SCDIs by differential formulation so as to transcriptionally modulate two or more distinct steps in the same multi-stage biological process involving a symphony of crosstalk between different cell types in the same target tissue.” It is understood, in this articulation, that signalling crosstalk between multiple cell types is involved downstream of an initiating transcriptional event.

Anatomically targeted precision can be functionally specified as the ratio of SCDI in the targeted tissue (e.g. skin, or lymph node) versus in the plasma tissue—specified, for instance, as nanograms of SCDI per milligram of total protein. Anatomical precision means that the ratio of SCDI in the targeted tissue versus in the plasma is >3 at some specified time point post-delivery, such as 2 hours; or at Cmax.

Immodulins are synthetic peptides that efficiently utilize iron-mediated cellular uptake, importin-mediated nuclear translocation and binding to retinoid X receptor to mediate transcriptional effects. The use of side-chain derivatized immodulins (SCDI) as tunable therapeutic agents is described in this specification. Rat biodistribution studies show that, when applied transdermally to rats using a nanoemulsion, immodulin peptides rapidly partition to skin tissue resulting in a ~9× enrichment relative to plasma even at distant skin locations. The recent publications cited above (Mascarenhas et al, 2025; Mascarenhas 2022), both of which are hereby incorporated by reference into this specification, show that side-chain derivatization with ligands of RXR heterodimeric partners can trigger unique bioactivities in cultured macrophages, CD4+ T-cells, dermal fibroblasts, muscle cells and keratinocytes. Transcriptional re-direction of circuits relevant to tissue repair, such as CD169+ macrophage differentiation, previously shown to drive differentiation of CD4+ Tcells into iTregs, can be increased by SCDIs with an RARα/β ligand. IL-19, IL-22 and IL-24 production by HaCaT keratinocytes and FGF7/KGF-1, TGFβ and COL1A1 by dermal fibroblasts, key components of a paracrine circuitry used in wound healing, can be amplified by an RARγ agonist or a partial PPARγ ligand. Myoblast differentiation is stimulated by a PPARα ligand. Versatile SCDI scaffolds hold promise for the rapid and inexpensive development of safe, targeted, self-administered therapeutic agents for skin.

In order to mitigate development costs, new drug design may attempt to triangulate the evolution of healthcare delivery systems, regulatory agencies' growing emphasis on safety, patent life considerations and emerging consumer preferences regarding treatment modalities. Such cross-currents saddle drug designers with the need to devise novel approaches. Historically, small-molecule drugs have been administered as pills or injectables in doses which are sufficient to flood the body. As a rule, these molecules lack guidance systems for precisely finding the anatomic locations that require their therapeutic action, leading to a plethora of off-target side effects. One favored conceptual approach to this problem is to marry the diversity achievable by combining old, clinically vetted chemical entities with the evolved biological “intelligence” of natural molecules.

So far, drug-targeting technologies have mainly homed to cell-surface targets, an approach of limited value for epigenetic drugs such as HDAC inhibitors and nuclear receptor ligands. In this study, we explore the use of small chemical molecules with known epigenetic bioactivity as side-chains of immodulin peptides. Immodulins are synthetic equivalents of natural protein sequences from a family of insulin-like growth factor binding proteins that direct the modulation of retinoid X receptor (RXR)—a master transcriptional regulator. Immodulins are capable of cell targeting, iron-mediated cell entry, nuclear translocation and direct binding to RXR. As such, they offer a scaffold for efficient linkage and delivery of small-molecule epigenetic modulators to a precisely identified transcriptional complex inside the cell nucleus of any mammalian cell. Hundreds of RXR-heterodimer-specific small-molecule modulators have been investigated, including PPAR and RAR ligands. Some, such as fenofibrate, tamibarotene and palovarotene have gained regulatory approval as drugs in their own right, despite their inherent targeting inefficiencies in vivo.

Emerging patient demands, have resulted in elevated compound average growth rates for products and services suited to “distance medicine”. These trends point to self-administered drug modalities such as transdermals, inhalants and nasal sprays. An unspoken requirement of such market evolution towards unsupervised patient drug use is the need for broader therapeutic indexes. In theory, the SCDIs explored in this study could align well with future therapeutic contexts if sufficient anatomical and mechanistic precision could be demonstrated. In this study, we use the biodistribution of immodulins in rats to demonstrate their surprising homing to skin tissue, and cultured skin cells to demonstrate the mechanistic specificity of SCDIs in major cell types found within the targeted tissue.

Therapeutic index (TI) is a measure of drug safety based on the differential between minimal effective dose and maximal tolerable dose. Many small-molecule drugs show limited TI (below 10). Given patient variability, such a limited range can become further narrowed for a drug in practice, complicating its real-world use. Epigenetic drugs such as nuclear receptor ligands (NRLs)—which must reach the target cell's nucleus in order to function—pose a particular challenge because small-molecule drugs lack the evolved biological “intelligence” that natural molecules employ in order to reach their intracellular targets efficiently. The observed TI of one FDA-approved NLR, bexarotene, is only about 3-4 when used to treat cancer in the lymphatic system. Methods and compositions are disclosed herein for the treatment of peripheral immune tissues (PIT) using NRLs covalently attached to immodulin peptides. Dramatic improvements in the TI of NRLs can be gained from the immodulin partner's tissue specificity (natural homing to PIT), precise organelle targeting (efficient cellular uptake and nuclear transport), and tunable mechanism of transcriptional action (immodulins themselves naturally drive intranuclear RXR heterodimer formation, thereby placing NRLs in close proximity to their RXR-partner targets). The combined effect of such improvements is a 2-100-fold improvement in TI for small-molecule NRL drugs. The combined effect of such improvements is a 2-100-fold improvement in functional TI for small-molecule NRL drugs. This invention discloses key components of that observed improvement: a preferential anatomical distribution of immodulins to the peripheral immune system when administered via a transdermal route, addition of hyaluronic acid to the diluent to enhance homing, and the novel use of transferrin for enhancing NRL-liganded immodulin recovery from a polymer-protected transdermal formulation.

Natural homing of immodulins to skin and surface lymphatics, while maintaining low to undetectable levels in general circulation, appears to be an active sequestration phenomenon, rather just a proximity phenomenon from transdermal administration to a particular anatomical location (Mascarenhas et al, 2025). In support of this claim, examples herein show that when the site of transdermal administration is the forearm (upper limb), high concentrations of fluorescent-labeled immodulin accumulate similarly in calf skin and draining lymph node (popoliteal) of the lower limb as well, with much lower concentrations in plasma. Conversely, when applied transdermally to calf skin, high concentrations of immodulin are also found in skin and draining lymph nodes (brachial) of the upper limb-again without generating comparable levels in plasma, blood cells or mesenteric lymph nodes. This type of biodistribution has not, to the inventor's knowledge, ever been reported for an epigenetic drug.

Immodulin peptides and ligand modifications thereof are described in PCT/US2023/013908, PCT/US2022/018449, PCT/US2021/046814, PCT/US2021/021433 and PCT/US2020/024828, as well as in recent scientific publications (Mascarenhas et al, 2025; Mascarenhas, 2022; and references cited therein). All these are hereby incorporated by reference.

The term ‘precision’, as used herein, refers to improved mechanistic, anatomical and temporal precision in the administration of SCDIs as medicine or cosmetic to a mammalian tissue in vivo or ex vivo.

This invention provides compositions and methods that substantially increase anatomic specificity of therapeutics to the peripheral immune tissues. As disclosed here for the first time, native immodulin peptides possess a capability for targeting these immune tissues, especially surface lymphatics of the skin. Transdermal administration of peptides, especially when trapped in protective polymers such as poly-lactic-co-glycolic acid (PLGA), introduces practical problems in the efficient release of immodulin peptides from the polymer. Disclosed in a recent scientific publication (Mascarenhas et al, 2025) is the surprising observation that mixing recombinant human holo-transferrin with the immodulin during encapsulation in a PLGA polymer increases release efficiency by more than three-fold. Transferrin is a natural ligand of immodulin peptides (Mascarenhas, 2022). The method of this invention therefore includes an optional step wherein transferrin is added to a ligand-modified peptide prior to encapsulation in a polymer. Another natural ligand, hyaluronic acid, is found to enhance efficacy of immodulin-based constructs in a rat burn model. This may reflect improved targeting to lymphatics in vivo. The method of this invention therefore includes the optional inclusion of hyaluronate in the diluent.

Covalent addition of small molecules to peptides and proteins is widely employed in laboratory research, but can be prohibitively expensive to manufacture at scale, as well as biologically problematic, for example, with respect to immunogenicity. Using a fragment of an abundant natural human protein as a scaffold for drug development has obvious advantages, such as the presumed benefit of over a billion years of natural evolutionary design providing biological intelligence that small-molecule drugs nearly always lack. Formulated SCDIs show dramatically precise anatomical targeting, protection from degradation in transit, rapid and selective uptake into cells within target organs, and precise transport within cells to the appropriate nuclear location and transcriptional complex for therapeutic action. Nuclear receptor ligands are examples of previously tested small-molecule drugs that can be repurposed with great specificity using the novel technologies disclosed in this invention. The resulting SCDIs are tunable transcriptional regulators that exquisitely modulate a cell's transcriptional machinery at will. They can be administered to a subject using a traditional route and can find their way to the desired target with far greater efficiency than the ligands alone.

Therapeutic index (TI) is a ratio that expresses the relationship between the dose expected to elicit some adverse effect (e.g., LD₅₀, TD₅₀, etc.) and the dose needed to elicit therapeutic effects (e.g., ED₅₀). The expansion of TI is critical to the expanded use of drugs in distance medicine e.g. involving needle-free self-administration of the drug by patients. This fits a powerful current trend in the evolution of healthcare delivery in advanced economies. Needle-free administration using transdermal and intranasal routes is thus relevant to the health industry.

Covalent addition of small-molecule drugs to antibodies (antibody-drug conjugates, or ADCs) has been explored as a method of expanding functional therapeutic index. Only surface markers (which represent <5% of all druggable targets) can be targeted. If the location for drug action lies inside the nucleus, ADCs may not be useful.

A recent scientific publication (Mascarenhas et al, 2025) reports biodistribution experiments in rats that show striking changes in tissue accumulation of SCDIs using different routes of administration. In particular, samples from the peripheral immune system, such as skin and draining lymph nodes of the skin, show enhanced concentrations of peptide, at 2 hours post transdermal administration, compared to traditional routes such as subcutaneous bolus injection or intranasal administration. Using hyaluronic acid as a carrier can accentuate this targeting effect.

In one embodiment, the invention provides a method wherein at least two hours after completion of agent delivery, the concentration of a peptide agent in the skin tissue of a limb located distant from the site of application is at least three-fold higher than the concentration of that agent in plasma. In a more preferred embodiment, the invention provides a method wherein at least two hours after completion of agent delivery, the concentration of a peptide agent in the skin tissue of a limb located distant from the site of application is at least five-fold higher than the concentration of that agent in plasma. In an even more preferred embodiment, the invention provides a method wherein at least two hours after completion of agent delivery, the concentration of a peptide agent in the skin tissue of a limb located distant from the site of application is at least ten-fold higher than the concentration of that agent in plasma.

Using amide bonds that resemble those naturally present in peptides and proteins for the covalent addition of small molecules to peptides is desirable. PCT/US2020/024828 teaches methods and compositions based on N-terminal covalent addition of non-amino acid carboxylate small molecule ligands to immodulin peptides, a conjugation step that occurs after assembly of the amino acid chain in synthesis. PCT/US2023/013908 teaches methods and compositions based on C-terminal covalent addition of non-amino-acid NLRs to immodulin peptides in a conjugation step that occurs prior to full amino acid chain elongation in synthesis. Each of these two approaches answers a different set of technical challenges that are industrially relevant to manufacture of immodulin-based targeted drugs at scale.

Recovery of therapeutics from widely employed biodegradable polymers such as poly-lactic-co-glycolic acid (PLGA) is often limited. This is thought to be a result of aggregation of the therapeutic molecule during the encapsulation process. Herein disclosed for the first time is the observation that mixing an immodulin with recombinant human holo-transferrin—which is readily available in GMP grade at reasonable cost from numerous suppliers—can improve the percentage of recovered therapeutic by more than three-fold. This is an example of the many practical uses of combined improvements described in this invention.

This invention discloses new compositions and improved utilities of the liganded-immodulin peptide class. Sequence extensions to the immodulin peptide sequences themselves can also confer extended biological activities useful in medicine and consumer products. Sequence extension of immodulin peptides has been described in PCT/US2020/024828, PCT/US2023/013908, PCT/US2022/018449 and PCT/US2021/046814, as well as in U.S. Pat. Nos. 5,519,003/5,783,405/6,165,977/6,262,023/6,342,368/6,423,684/6,855,693/6,933,275/7,393,835/8,536,135/10,369,1919 and references cited therein.

In one embodiment, the invention provides a method for modulating a multi-stage biological process by delivering a first and a second agent to a living mammalian tissue in vivo or ex vivo, each agent being a side-chain-derivatized immodulin peptide 20-60 amino acids long comprising an amino acid sequence corresponding to SEQ ID NO: 1 or SEQ ID NO:2 and further comprising a covalently attached nuclear receptor ligand, wherein said first and second agent are each attached to a different ligand and peak delivery of first agent is at least four hours (or, more preferably, twelve hours) before peak delivery of second agent and the tissue concentration of agent two hours after delivery is at least two-fold (or, more preferably three- or five- or ten-fold) higher in a skin tissue taken from a limb distant from the site of application than the concentration of agent in the plasma.

In a preferred embodiment, the invention provides a method for improving a multi-stage biological process by delivering to the skin of a mammal via transdermal or intradermal route a first and a second synthetic side-chain-derivatized immodulin peptide 20-60 amino acids long and comprising an amino acid sequence corresponding to either SEQ ID NO:1 or SEQ ID NO:2, wherein:
(a) each peptide is covalently attached to one or more ligands for nuclear receptors selected from a group consisting of RARα, RARβ, RARα/β, RARβ/γ, RARγ, PPARα, PPARβ, PPARδ, PPARγ, LXRβ, LXRα, FXRα, RXRα and RXRγ;
(b) plasma Cmax for first agent is reached at least four hours prior to plasma Cmax for second agent;
(c) peak production of at least one Tier A protein occurs at least four hours prior to peak production of at least one Tier B protein in skin tissue; wherein
(d) Tier A proteins are selected from a group consisting of CD169, CCL22, IL-10, and FoxP3; and
(e) Tier B proteins are selected from a group consisting of IL-19, IL-22, IL-24, FGF7, COL1A1 and creatine kinase;
   thereby significantly improving the orderly execution of said multi-stage biological process.

In a preferred embodiment, the invention provides a method for modulating a multi-stage biological process by delivering a first and a second peptide to skin tissue wherein (a) first synthetic side-chain-derivatized immodulin peptide is covalently attached to one or more ligands for nuclear receptors selected from a group consisting of RARα, RARβ, RARα/β, RARγ, RXRα and RXRγ; and (b) second synthetic side-chain-derivatized immodulin peptide is covalently attached to one or more ligands for nuclear receptors selected from a group consisting of RARγ, PPARα, PPARγ, RXRα and RXRγ; and (c) the tissue concentration of each agent two hours after delivery is at least two- to ten-fold higher in a skin tissue from a limb distant from the site of application than the concentration in plasma.

In some embodiments, the nuclear-receptor-ligand-modified peptide is formulated with purified recombinant human transferrin and trapped in a biodegradable polymer formulation;

In some embodiments, the nuclear-receptor-ligand-modified peptide is formulated with purified recombinant human transferrin at a peptide-to-transferrin molar ratio of between 0.01 and 50.

In some embodiments, the nuclear-receptor-ligand-modified peptide is formulated in a suitable pharmaceutical diluent containing at least 0.001% high-molecular-weight hyaluronic acid;

In some embodiments, the nuclear-receptor-ligand-modified peptide is administered to a mammalian subject via a transdermal or intradermal route;

In some embodiments the invention provides a liganded immodulin peptide wherein the small-molecule nuclear receptor ligand of mass less than about 1000 daltons is selected from a group consisting of HX600, HX630, celastrol, LG100754, GW7647, tamibarotene, GW3965, AM580, palovarotene, adapalene, bexarotene, capric acid, fenofibric acid, GW4064, sobetirol and GW501516.

In some embodiments, the diluent used in the administration of the liganded peptide contains at least 0.001% of high-molecular-weight hyaluronic acid and/or a botanical compound selected from a group consisting of resveratrol, quercetin, menthol, anisic acid, verbenone, eugenol, linalool, ferulic acid, glycyrrhizic acid, levulinic acid, alkali salt of olive oil and alkali salt of coconut oil.

In some embodiments the liganded peptide is complexed with recombinant human holo-transferrin and encapsulated in a biodegradable polymer such as poly-lactic-co-glycolic acid (PLGA) or polycaprolactone (PCL).

In yet another related aspect, the invention provides a method for treating a subject suffering from immunological, neurological, oncologic, skeletal, reproductive, metabolic or cosmetic dysfunction or perturbation, where the method includes administering to the subject (e.g., a human subject) a therapeutically effective dose of SCDI peptide, peptide complex, or other pharmaceutical composition described in the invention. In some embodiments the therapeutically effective dose of SCDI peptide is from about 0.001 mg/kg/day to about 50 mg/kg/day.

In yet another related aspect, the invention provides an in vitro method for measuring the potency of any immodulin peptide described herein using cultured mammalian cells.

### Definitions and Detailed Description

This invention teaches a method for sequential control-release of specified SCDIs (side-chain-derivatized immodulins) in order to govern the sequential production of defined protein sets within the context of a multi-stage biological process in skin tissue in vivo or ex vivo.

“Two distinct SCDIs” means two immodulin peptides whose side chains have been covalently attached to different NR ligands. It is understood that a NR ligand modulates its cognate NR.

The invention discloses a method for modulating a multi-stage biological process by delivering to the skin of a mammal a first and a second synthetic side-chain-derivatized immodulin peptide 20-60 amino acids long and comprising an amino acid sequence corresponding to either SEQ ID NO:1 or SEQ ID NO:2, wherein (a) each peptide is covalently attached to one or more nuclear receptor ligands selected from a group consisting of TRα, TRβ, RARα, RARβ, RARα/β, RARβ/γ, RARγ, PPARα, PPARβ/δ, PPARγ, REV-ERBα, REV-ERBβ, RORα, RORβ, RORγ, LXRβ, LXRα, FXRα, VDR, PXR, CAR, HNF4α, HNF4γ, RXRα, RXRβ, RXRγ, TR2, TR4, TLX, PNR, COUP-TFα, COUP-TFβ, COUP-TFγ, ERα, ERβ, ERRα, ERRβ, ERRγ, MR, PR, GR, AR, Nur77, NURR1, and NOR-1 ligands; (b) sequential release of first and second agents involves plasma Cmax of first agent occuring at least four hours prior to plasma Cmax of second agent; and wherein (c) at least two hours after administration the concentration of agent in the skin tissue of a limb located distant from the site of application is at least three-fold higher than the concentration of that agent in plasma tissue.

“A multi-stage biological process” is an orderly stepwise sequence characterized by distinctive transcriptional events and the expression of downstream proteins such as cell surface markers and cytokines. Most processes within living tissues fit this description. Some particularly relevant processes include the legislation of inflammatory events and their resolution in tissues, and other healing processes—notably in skin compromised by ageing, environmental insult, autoimmune conditions or diabetes, and in wounded skin.

“Delivering to the skin” means delivering peptides by transdermal or intradermal or other route where the primary end-target tissue is skin. Most cosmetic and dermatological routes of delivery are included in this definition because the primary end-target tissue is skin.

“Side-chain-derivatized immodulin peptides” or SCDIs are immodulin peptides (Mascarenhas 2022) derivatized by covalent addition of small molecules at or near their N- or C-termini, or both. Many examples, and methods for making SCDIs, can be found in Mascarenhas et al, 2025.

In a preferred embodiment, the first and second peptide agents are released to the tissue sequentially. This can be accomplished by formulations that restrict the release of the two agents in some temporally different manner (as shown in Example 4), or using routes of delivery that delay the availability of one agent relative to the other, or it can also mean application of the second agent after some time interval has elapsed following the application of the first agent, or a combination of more than one of these approaches.

In a preferred embodiment, the peptides of the invention comprise an amino acid sequence corresponding to either SEQ ID NO:1 or SEQ ID NO:2. It should be understood that the scope of this statement includes extensions, mutations and analogs of these amino acid sequences.

In one embodiment, the peptides are covalently linked to nuclear receptor ligand for one or more nuclear receptors (NRs). The following are specifically covered by this invention. In each case the NR protein name is given, followed by the NR genomic class: TRα (NR1A1), TRβ (NR1A2), RARα (NR1B1), RARβ (NR1B2), RARγ (NR1B3), PPARα (NR1C1), PPARβ/δ(NR1C2), PPARγ (NR1C3), REV-ERBα (NR1D1), REV-ERBβ (NR1D2), RORα (NR1F1), RORβ (NR1F2), RORγ (NR1F3), LXRβ (NR1H2), LXRα (NR1H3), FXRα (NR1H4), VDR (NR1I1), PXR (NR1I2), CAR (NR1I3), HNF4α (NR2A1), HNF4γ (NR2A2), RXRα (NR2B1), RXRβ (NR2B2), RXRγ (NR2B3), TR2 (NR2C1), TR4 (NR2C2), TLX (NR2E1), PNR (NR2E3), COUP-TFα (NR2F1), COUP-TFβ (NR2F2), COUP-TFγ (NR2F6), ERα (NR3A1), ERβ (NR3A2), ERRα (NR3B1), ERRβ (NR3B2), ERRγ (NR3B3), MR (NR3C2), PR (NR3C3), GR (NR3Cl), AR (NR3I4), Nur77 (NR4A1), NURR1 (NR4A2), NOR-1 (NR4A3) and heterodimers thereof. Examples of heterodimers include RARα/β and RARβ/γ. In a preferred embodiment, the peptides are covalently linked to one or more NRs selected from the following list: TRα, TRβ, RARα, RARβ, RARα/β, RARβ/γ, RARγ, PPARα, PPARβ/δ, PPARγ, REV-ERβα, REV-ERββ, RORα, RORβ, RORγ, LXRβ, LXRα, FXRα, VDR, PXR, CAR, HNF4α, HNF4γ, RXRα, RXRβ, RXRγ, TR2, TR4, TLX, PNR, COUP-TFα, COUP-TFβ, COUP-TFγ, ERα, ERβ, ERRα, ERRβ, ERRγ, MR, PR, GR, AR, Nur77, NURR1 and NOR-1.

One distinctive feature of the invention is anatomical privileging of peripheral tissues such as skin and surface lymphatics. As shown below, this natural “homing” feature of immodulins (disclosed in Mascarenhas et al, 2025) enables exceptional anatomical precision of action in skin and surface lymphatic tissues. In a preferred embodiment the method of the invention claims substantial enrichment of peptide agents in skin, two hours after Cmax. Here, “Cmax” refers to the maximum concentration of the agent achieved in blood after a dose is applied. “Two hours after Cmax” means two hours after the Cmax concentration has begun to trend lower from its peak. Concentrations of peptide agent in blood and tissues is expressed as weight of peptide established by common analytics such as ELISA assay (or deduced from transcriptomic analysis) per milligram of total plasma or tissue protein. In one embodiment, the concentration of peptide in skin is 3× the concentration in plasma. In a more preferred embodiment.

“Selectively modulating accumulation of key proteins in tissue in a manner beneficial to a multi-stage biological process” means modulating by 30% or more the levels of at least three key mRNA transcripts (or their protein products) associated with a desired biological response in the tissue. A “key” transcript or protein is one so designated in the context of the desired biological response by virtue of evidence published in one or more peer-reviewed scientific papers. Beneficial modulation may involve up-regulating or down-regulating such a transcript, or its protein product, in a manner that is desired in order to modulate the targeted multi-stage biological process.

“Therapeutic index” (TI) is a ratio that expresses the relationship between the dose expected to elicit some adverse effect (e.g., LD₅₀, TD₅₀, etc.) and the dose needed to elicit therapeutic effects (e.g., ED₅₀).

The invention comprises a method of transcriptional choreography for selectively modulating a multi-stage biological process by contemporaneously administering to the skin or surface lymphatic tissue of a mammal a first and a second synthetic side-chain-derivatized immodulin peptide 20-60 amino acids long and comprising an amino acid sequence corresponding to either SEQ ID NO:1 or SEQ ID NO:2 covalently attached to a ligand for a RAR, PPAR or RXR nuclear receptor, wherein:
(a) the ligand covalently attached to first peptide is different from the ligand covalently attached to second peptide; and
(b) first peptide is formulated and administered so the peak concentration of first peptide in tissue is achieved at least four hours prior to when peak concentration of second peptide is achieved in the same tissue; and
(c) peak accumulation of CD169 protein occurs prior to peak accumulation in the same tissue of a protein selected from a group consisting of IL-19, IL-22, IL-24, and FGF7 and COL1A1;
thereby selectively modulating said multi-stage biological process.

“Contemporaneously administering” means administering a single dose containing multiple elements or separate administration within a contemporaneous time frame. Contemporaneously is functionally defined as substantially similar in effect to administering multiple elements in a single dose.

“Modulating” can apply to increasing or decreasing the concentration of the desired substance in tissue. “Multi-stage” means two or more stages.

Peak accumulation or concentration of an RNA or protein in tissue means the concentration of that molecule, as measured by suitable assay of tissue extracts. Peak differential accumulation or concentration means the concentration difference between treated tissue untreated control tissue.

The terms “subject” and “individual”, as used herein, refer to mammalian individuals, and more particularly to domesticated animals (e.g., dogs, cats, and the like), agricultural animals (e.g., cows, horses, sheep, and the like), and primates (e.g., humans).

The terms “liganded immodulin peptide” or “SCDI” means a peptide molecule 20-60 amino acids in length prepared by chemical synthesis and comprising an immodulin peptide sequence corresponding to SEQ ID NO: 1 or SEQ ID NO:2 or homologs and variants thereof, further modified by covalent attachment to at least one nuclear receptor ligand.

“RXR” means retinoid X receptor, and can refer to either the RXR gene or the protein it specifies. “Rexinoid” means a ligand of an RXR receptor. “RXRs” means any of the RXR isoforms, such as RXR-alpha (RXRα), RXR-beta (RXRβ), RXR-gamma (RXRγ), which may form heterodimers with other nuclear receptors (NRs) in their own class, or in other classes.

RXRs are known to form heterodimers with members of several classes of NRs. NRs are classified into seven classes, NR0 through NR6. It is known that classes NR1—NR4 contain heterodimeric partners of RXRs. Each class contains subclasses e.g. class NR1 contains NR1A, NR1B, NR1C, NR1D, NR1F, NR1H and NR1I. Members of at least 5 of these subclasses can form heterodimers with RXRs. Members of NR2, NR3 and NR4 classes also contain members capable of forming heterodimers with RXRs. RXR receptors can form functional heterodimers with efficiencies that vary with RXR isoform, tissue distribution and nuclear concentrations of each nuclear receptor under a given biological context, the action of epigenetic modulators, and many other variables.

Some nuclear receptor classes of particular interest include:

| Class | Protein |
|---|---|
| NR1A1 | TRα |
| NR1A2 | TRβ |
| NR1B1 | RARα |
| NR1B2 | RARβ |
| NR1B3 | RARγ |
| NR1C1 | PPARα |
| NR1C2 | PPARβ/δ |
| NR1C3 | PPARγ |
| NR1D1 | REV-ERBα |
| NR1D2 | REV-ERBβ |
| NR1F1 | RORα |
| NR1F2 | RORβ |
| NR1F3 | RORγ |
| NR1H2 | LXRβ |
| NR1H3 | LXRα |
| NR1H4 | FXRα |
| NR1I1 | VDR |
| NR1I2 | PXR |
| NR1I3 | CAR |
| NR2A1 | HNF4α |
| NR2A2 | HNF4γ |
| NR2B1 | RXRα |
| NR2B2 | RXRβ |
| NR2B3 | RXRγ |
| NR2C1 | TR2 |
| NR2C2 | TR4 |
| NR2E1 | TLX |
| NR2E3 | PNR |
| NR2F1 | COUP-TFα |
| NR2F2 | COUP-TFβ |
| NR2F6 | COUP-TFγ |
| NR3A1 | ERα |
| NR3A2 | ERβ |
| NR3B1 | ERRα |
| NR3B2 | ERRβ |
| NR3B3 | ERRγ |
| NR3C2 | MR |
| NR3C3 | PR |
| NR3CI | GR |
| NR3I4 | AR |
| NR4A1 | Nur77 |
| NR4A2 | NURR1 |
| NR4A3 | NOR-1 |

This invention also envisages an in vitro assay method for measuring the biological potency or biodistribution of an immodulin peptide. As will be understood by those of skill in the art, the mode of detection of a diagnostic signal will depend on the exact detection system utilized in the assay. For example, if a fluorescent detection reagent is utilized, the signal will be measured using a technology capable of quantitating the signal from the sample, such as by the use of a fluorometer. If a chemiluminescent detection system is used, then the signal will typically be detected using a luminometer. Methods for assaying fluorescent or biological signals are well known in the art. Many assay methods are shown in Mascarenhas et al, 2025 and references therein.

It will be readily apparent to a practitioner skilled in the art that sequences claimed hereunder include homologs, mutants and other variants of an immodulin peptide-including by conservative substitution, extension or deletion in amino acid sequence. Trivial substitution variants include swapping of an amino acid with another belonging to the same class, without such substitution resulting in any significant and measurable functional improvement. “Classes” of amino acids include positively charged amino acids (arginine, lysine, histidine), negatively charged amino acids (aspartic acid, glutamic acid), aromatic amino acids (tyrosine, phenylalanine, tryptophan), branched chain amino acids (valine, leucine isoleucine) and other natural groupings such as (serine, threonine) and (asparagine, glutamine).

As will be understood by those of skill in the art, the multi-stage biological responses modulated by the methods described herein, as well as the methods used to measure outcomes may vary, depending on a particular disease or individual subject. All references cited in this document, including Mascarenhas et al, 2025 and Mascarenhas 2022, all cited patent applications and the references cited therein, are incorporated by reference in their entirety.

### EXAMPLES

Example 1. Modification of peptide with nuclear receptor ligands of low molecular mass. N-terminal modification of peptides with biotin, or fatty acids such as myristic and palmitic acids, is widely known in the field. Other types of carboxylic acids have rarely been used in this way, possibly due to technical difficulties in recovering the correct product. For immodulin peptides, previous disclosures of N-terminal modification using non-conventional compounds include PCT/US2022/018449, PCT/US2021/046814, PCT/US2021/021433 and PCT/US2020/024828 (now issued U.S. patent Ser. No. 12/102,685), as well as scientific publications (see, for example, Mascarenhas et al, 2025; Mascarenhas, 2022; and references cited therein). PCT/US2020/024828 teaches methods and compositions based on N-terminal covalent addition of non-amino acid carboxylate small molecules to immodulin peptides, a conjugation step that occurs after assembly of the amino acid part of the chain in synthesis. Sufficient molar ratios of the non-amino acid carboxylate must be used in that process, in order to satisfy not just a reaction with the correctly assembled chain, but also the much more numerous failure sequences (such as deletions) accumulated during chain elongation. For immodulin peptides, which can typically be between 35-45 amino acid residues in length (and often longer), such failure sequences can significantly raise the cost of identification and recovery of useful product from the reaction mixture. In practice, therefore, the use of the N-terminal addition method disclosed in PCT/US2020/024828 is largely limited by the cost of the said non-amino acid carboxylate reagent. The covalent addition of non-amino acid carboxylates to a C-terminally located lysine in the immodulin peptide sequence poses other challenges. This coupling occurs near the start of amino acid chain elongation during synthesis. Regardless of the length of the peptide chain, the option to couple a non-amino acid carboxylate to the epsilon amino group of a C-terminal lysine monomer via a covalent bond obviates the abovementioned technical problems encountered with the method disclosed in PCT/US2020/024828, vastly increasing the options previously foreclosed due to cost. In short, C-terminal addition allows for the addition of more expensive compounds. As described, the invention provides for the addition of non-amino acid carboxylates wherein said molecules can bind a nuclear receptor with sub-millimolar affinity in vitro. There are, however, novel technical issues that limit C-terminal in-synthesis conjugation of such non-amino acid carboxylates. For example, said compounds must be stable enough to survive multiple cycles of synthesis under the harsh conditions typically employed for automated peptide synthesis. For some compounds, if the conjugation is not efficient, the C-terminal monomer may need to be modified and pre-purified prior to use in chain elongation. The general methods disclosed herein are applicable to a number of compounds that are capable of binding a nuclear receptor with sub-millimolar affinity in vitro. In practice, however, it is not always possible to predict in advance which ligands will work satisfactorily, and which will not. This method can be used to couple small nuclear receptor ligands to the C-terminus of an immodulin peptide during synthesis. The peptides were synthesized on ChemMatrix Rink Amide resin, as follows, using a modified Fmoc synthesis protocol with DIC/CI-HOBt coupling, on an APEX 396 automatic synthesizer. For example, using GW7647 as the compound, Fmoc-d-Lys (GW7647)-OH was customized in this way: GW7647 was activated as an NHS-ester and then reacted with Fmoc-d-Lys-OH, which had a free epsilon amine on its side chain. The FMOC-d-Lys (GW7647) wang resin was swollen in dimethylformamide (DMF) for 30 min, treated with 20v % Piperidine-DMF for 8 minutes to remove the Fmoc protecting group at 50° C., and washed with DMF three times. For the coupling reaction, the resin was added with Fmoc-protected amino acid, CI-HOBt, DIC and NMP. The mixture was vortexed for 20 minutes at 50° C. Afterwards, the resin was washed with DMF once. The cycle of deprotection and coupling steps was repeated until the last biotin residue was assembled. The resin was then washed with DMF, DCM and dried with air. The peptide was cleaved from the resin using a TFA cocktail (95v % TFA, 2.5v % water and 2.5v % TIS) for three hours. Crude peptides were precipitated by adding ice-chilled anhydrous ethyl ether, washed with anhydrous ethyl ether for three times, and dried in vacuo. The purification was performed accordingly by a prep-HPLC. The results of the conjugation experiments show that there is wide variation in conjugation efficiency from compound to compound. As the practicality and cost of synthesis can be dramatically affected when product yield is low, it is therefore not obvious a prior that any untested carboxylic acid should be assumed to be a good candidate for this type of peptide modification. The use of compounds tested here and listed in the table below has never been reported for this kind of peptide modification. It appears that that chance of practical success is low until shown. Once shown, however, the C-terminal modification of an immodulin peptide with a given compound is nearly always highly reproduceable. Some examples are shown below:

TABLE X1.1
Some nuclear receptor ligands.

| Ligand Class | Compound | CAS No. | MW |
|---|---|---|---|
| PPARα | GW7647 | 265129-71-3 | 502.8 |
| PPARα | Fenofibric acid | 42017-89-0 | 318.8 |
| PPARd | GW501516 | 317318-70-0 | 453.5 |
| PPARγ | Capric acid (decanoate) | 1002-62-6 | 194.2 |
| FXR | GW4064 | 278779-30-9 | 542.8 |
| LXR | GW3965 | 405911-17-3 | 618.5 |
| RARβ/γ | Adapalene | 106685-40-9 | 412.5 |
| RARα/β | Tamibarotene (AM80) | 94497-51-5 | 351.4 |
| RARα | AM580 | 102121-60-8 | 351.4 |
| RARβ | BMS453 | 166977-43-1 | 380.5 |
| RARγ | Palovarotene | 410528-02-8 | 414.5 |
| RXR | Bexarotene | 153559-49-0 | 348.5 |
| RXR | Celastrol | 34157-83-0 | 450.6 |
| RXR | HX600 | 172705-89-4 | 438.6 |
| RXR | LG100754 | 180713-37-5 | 396.6 |

Practitioners well versed in the art will recognize the possibility of adding NR ligands to the N-terminus of a carboxyterminally liganded-immodulin, using the methods for N-terminal modification referenced above. For example, ligand GW4064 can be added to the N-terminus of immodulin peptide coupled to bexarotene at the C-terminus. Examples have been recently published (Mascarenhas et al, 2025). This type of construction allows two different ligands to modify transcription within the same cell nucleus simultaneously. Adding the two ligands separately to achieve a similar dual-transcriptional result in a live mammal would not be practicable because of likely dose-limited toxicities at the high doses that would be required.

Biological effects of liganded immodulins can be shown in many different biological systems, both in vitro and in vivo. Mascarenhas et al (2025) and Mascarenhas (2022) provide detailed descriptions of assays for CD169+ macrophage generation and subsequent cell-cell effects of these macrophages on T-helper (Th) subsets by co-culture with CD4+naïve T-helper cells. Briefly, macrophage differentiation and polarization assays were done using the THP1-Dual monocyte reporter cell line (Invivogen Inc, San Diego, CA) seeded at 2×10e5 cells per well in 96-well plates and cultured at 37 degrees C. in RPMI-1640 growth medium plus 10% fetal bovine serum and 1% penicillin/streptomycin, then treated for 24 hours with 100 ng/ml Phorbol 12-myristate 13-acetate (macrophage PMA protocol). Peptide (330 or 660 nM) was then added, and incubation continued for an additional 24 hours. Culture supernatants were then assayed for C-C motif chemokine 22 (CCL22), interleukin-10 (IL-10) or transforming growth factor beta (TGFbeta) using Duoset ELISA kits (R&D Systems, Minneapolis, MN). Adherent cells were washed twice with PBS and cells were assayed for immunoreactivity of surface markers CD169, Clec9a and Clec12a using biotin-labeled anti-human antibodies for these markers purchased from Miltenyi Biotec (Auburn, CA) and a streptavidin-horseradish peroxidase/TMB secondary detection reagent. Results were expressed as arbitrary ELISA immunoreactivity units relative to the control peptide (=100). For T-cell differentiation co-culture assays, the THP1-Dual monocyte reporter cell line was treated with PMA for 24 hours as described above, adherent cells were washed twice with PBS and approximately 2×10e5 naïve CD4+ T-cells (Zen-Bio Inc, Durham, NC) were layered on the macrophages in 100 uL RPMI-1640 containing peptide and 15 uL T-activator CD3/CD28 Dynabeads (Thermofisher Scientific, Waltham, MA) and incubated for a further 96 hours. Protein was assayed using the BCA protein assay kit from Thermofisher Scientific (Waltham, MA).

C2C12 myoblast cell line (ATCC, Manassas, VA) was cultured in DMEM medium supplemented with 10% FBS and 1% penicillin/streptomycin. For differentiation, cells were cultured in the same medium, except that 10% FBS was replaced by 2% horse serum. Peptides and NR ligands were added at the indicated concentrations and cells were harvested 96 hours later for assay. Cell extracts were assayed for creatine kinase (CK) per mg protein. CK was measured using the ECPK-100 kit from BioAssay Systems (Hayward, CA). Protein was assayed using the BCA protein assay kit from Thermofisher Scientific (Waltham, MA). By addition of an additional NR ligand to the C-terminus of an liganded-immodulin using the methods described above, it is possible to combine the effects of multiple transcriptional pathways at will. The performance of dual-ligand SCDIs is expected to be technically and industrially superior, owing to dose-related and regulatory advantages.

Example 2. Biodistribution of immodulins in vivo. Protocols and results of similar experiments are described in the recently published paper (Mascarenhas et al, 2025). In an ideal scenario, drug accumulates at therapeutic levels at a desired tissue location in the body while simultaneously exhibiting low levels in plasma. Therapeutic peptides can be encapsulated in PLGA or PCL microparticles or nanoparticles by a number of protocols widely known in the art. For encapsulation in microparticles containing immodulin peptides, the following protocol can be used: All solutions are filter-sterile. PLGA-M solution contains 50 mg/ml poly-lactide-co-glycolide (acid terminated) i.e. PLGA-COOH (Cat. #26268-1, Polysciences Inc, Warrington, PA) dissolved in acetone containing 4 mg/ml menthol. Microparticles are assembled as follows: 50 uL (100 μg) fluorescent-tagged-peptide (or HRP enzyme-tagged-peptide) solution is added to 75 μl saline buffer (FITC) or 75 μl recombinant human holo-transferrin (FITC-Tf). 1 ml PLGA-M solution is then added and the mixture is vortexed thoroughly. 2 ml of 2% polyvinyl alcohol is added, followed by vortexing. The resulting mixture is then added to 50 ml tubes containing 20 ml sterile distilled water and vortexed again. The tube is shaken at room temperature with the cap off for 2-4 hours to allow evaporation of the solvent. Particles are collected by centrifugation at 4,000 rpm in a Beckman centrifuge for 10 minutes. Particles may optionally be coated by resuspension in 500 uL of 2 mg/ml glycol chitosan (or other chitosan compound) for 30 mins at RT, then 500 uL of 10 mg/ml human serum albumin (HSA) is added and incubation at RT continued for 30 mins, followed by centrifugation. Finally, the particles are washed 3 times with 1 ml sterile distilled water and collected by centrifugation. In order to deliver the microparticles, they may be suspended in 10×NPDF nanoemulsion for transdermal delivery, 1×NPDF in saline for intranasal delivery, or saline for sub-cutaneous bolus delivery. 10×NPDF is prepared as follows: Base solvent is made by mixing 2 ml sterile distilled water, 4 ml propylene glycol, 16 ml diethylene glycol monoethyl ether and 16 ml glycerol. The following are dissolved in base solvent: 5 mM each menthol, p-anisic acid and verbenone; 1 mM each linalool, ferulic acid and glycyrrhizic acid. Finally, 25 ml of this solution in base solvent is added to 25 ml of 6 mM sodium acetate pH 5.2 containing 0.9% sodium chloride, and 10 mM levulinic acid. By using immodulin peptides with or without PLGA encapsulation, significantly increased delivery to tissues using microparticles suspended in the above diluents has been demonstrated. Addition of hyaluronic acid to the diluent increases delivery to target tissues. Biodistribution of 50 uL each prep in rats (n=4 to 8 per group) is scored as fluorescent counts (or enzymatic units) per milligram tissue protein. Using this metric, the following results were obtained at 2 hours post-administration (normalized to plasma=1.0). Legend: Naked (SQn) and PLGA-encapsulated (SQ) immodulin peptide in 50 uL PBS buffer via subcutaneous bolus; IN=PLGA-encapsulated immodulin peptide in 50 uL 1×NPDF-saline; TDc (calf) or TDf (forearm) PLGA-encapsulated HRP-labeled peptide in 50 uL 10×NPDF applied transdermally. All values shown are relative to the internal plasma control values i.e. showing relative biodistribution. Each tissue group is the average of five representative tissues in each group (see legend below table for details):

TABLE X3.1
Biodistribution of labeled immodulin peptides administered to rats via various routes.

| Admin Route | SQn ^{#} | SQ ^{#} | IN ^{#} | SQ ^{##} | TDc ^{##} | TDf ^{##} |
|---|---|---|---|---|---|---|
| Plasma | 1.00 ± 0.65 | 1.00 ± 0.05 | 1.00 ± 0.35 | 1.01 ± 0.00 | 1.00 ± 0.07 | 1.00 ± 0.23 |
| Peripheral | 1.91 ± 1.60 | 4.28 ± 0.86 | 3.95 ± 0.52 | 3.08 ± 0.75 | 10.12 ± 3.32 | 10.11 ± 1.55 |
| Immune Group | | | | | | |
| Muscle Group | 2.05 ± 0.31 | 2.83 ± 0.46* | 1.83 ± 0.55** | 2.62 ± 0.77 | 1.34 ± 0.13* | 2.61 ± 0.44** |
| Splanchnic Gp | 2.15 ± 0.35 | 7.39 ± 3.82 | 6.67 ± 1.31** | 9.50 ± 4.42* | 2.83 ± 1.33* | 4.21 ± 1.61** |
| T.I. ratio ^{&} | 0.61 | 1.04 | 1.42 | 1.00 | 6.40 | 3.30 |
| T.I. ratio ^{&&} | 1.53 | 1.00 | 1.02 | 1.00 | 11.05 | 7.42 |

^{#} FITC label;
^{##} HRP label;
*p < 0.05
**p < 0.01 versus peripheral immune group;
Peripheral Immune Group (average): forlimb skin, back skin, calf skin, brachial lymph node, popliteal lymph node;
Muscle Group (average): biceps, triceps, calf EDL, soleus, flexor ulnaris;
Splanchnic Group (average): Small Intestine, kidney, heart, liver, pancreas;
T.I. ratio:
^{&} = peripheral immune versus muscle group;
^{&&} = peripheral immune versus splanchnic group;
SQ = 1;

The results show a dramatic relative predicted expansion of functional T.I. when immodulin peptides are administered transdermally to upper or lower limb. In both cases, concentrations of peptide are enriched in both upper and lower limb peripheral lymphatic tissues (skin and surface draining lymph nodes), without corresponding increases in the plasma levels or in deep mesenteric lymph nodes (latter control was done, but is not shown). These results, taken together, suggest an active biological partitioning of immodulins to the peripheral lymphatic circulation. For additional details on this type of experiment, please see Mascarenhas et al, 2025.

### Example 3. Improved Release of Immodulins from Microparticles Using Transferrin

Functional TI can be further improved by using pharmaceutically acceptable formulations. Encapsulation of drugs in biodegradable PLGA microparticles is a widely employed technique in FDA-approved therapeutics, typically enhancing delivery characteristics of a drug. Actual use of PLGA encapsulation can be hampered by poor release of trapped drug. Surprisingly, co-encapsulation of immodulin-based drugs with recombinant human holo-transferrin results in a striking improvement in the release of immodulin peptides from PLGA particles. The use of transferrin to facilitate release of a peptide drug from PLGA particles is new. PLGA microparticles were prepared as described in Example 2. FITC-labeled peptide (FAM) was packaged either with or without transferrin. Release in NPDF diluent was measured by counting fluorescence in supernatants at 488 nm. Added transferrin improves release kinetics from microparticles by 300-400%. For additional details on this type of experiment, please see Mascarenhas et al, 2025.

TABLE X4.1
FITC-labeled immodulin peptide (FAM) with or without transferrin (Tf):

| | Hours | | | | | | |
|---|---|---|---|---|---|---|---|
| | 0.0 | 0.4 | 1.4 | 3.4 | 8.1 | 14.5 | 18.5 |
| FAM | 2.3 ± 1.7 | 3.5 ± 0.9 | 5.7 ± 2.8 | 6.2 ± 1.3 | 8.8 ± 1.0 | 11.2 ± 1.4 | 13.9 ± 0.9 |
| FAM + Tf | 25.1 ± 1.1* | 22.3 ± 2.2* | 26.5 ± 0.9* | 30.8 ± 2.2* | 36.1 ± 2.0* | 42.1 ± 2.9* | 52.5 ± 3.3* |

Percent released from PLGA microparticles in NPDF at 37 degrees C. over time.
*p < 0.05 peptide + transferrin versus peptide alone.

### Example 4. Dual Packaging and Non-Simultaneous Release of SCDIs from Microparticles Using PLGA or PCL Polymers

Encapsulation of drugs in biodegradable PLGA or PCL microparticles is a widely employed technique in FDA-approved therapeutics, typically enhancing delivery characteristics of a drug. PLGA or PCL microparticles were prepared as described in Example 2. PCL microparticles were prepared identically to PLGA particle except that 50 mg/ml PCL was used instead of 50 mg/ml PLGA in the encapsulation step. Fluorescently-labeled peptide and transferrin were co-packaged as described in Example 2, using different fluorophores for PLGA and PCL microparticles. This allowed direct comparison of release kinetics by side-by-side measurement at the relevant wavelengths (543 nm and 594 nm for PLGA and PCL particles respectively). Both PLGA and PCL microparticles were provided concurrently. Release was measured by counting fluorescence in supernatants in a fluorometer using 543 nm and 594 nm to track release from PLGA and PCL particles respectively. Rate of release per hour was calculated. The results are shown below (fluorescence shown as rate/hr in arbitrary units, background subtracted):

| | Hours | | | | | | | |
|---|---|---|---|---|---|---|---|---|
| | 0.0 | 0.5 | 1.5 | 13 | 14 | 15 | 16 | 17 |
| 543 nm (PLGA) | 15.4 | 260.5 | 82.6 | 60.8 | 25.2 | 0.0 | 0.0 | 0.0 |
| 594 nm (PCL) | 0.0 | 0.0 | 0.0 | 0.0 | 158.8 | 272.4 | 0.0 | 0.0 |

The results show a peak release of the encapsulated SCDI/transferrin at 0.8 and 14.7 hours for PLGA and PCL particles respectively. This demonstrates that sequential release of more than one agent can be achieved in a single dose of a formulation delivered to a living mammalian tissue in vivo or ex vivo by mixing two or more delivery modalities into a single dose.

### Example 5. Temporally Precise Ordering of Transcriptional Effects in Skin Cells

Additional data (Mascarenhas et al, 2025) have been gathered to show that side-chain derivatization with ligands of RXR heterodimeric partners can trigger unique bioactivities in cultured macrophages, CD4+ T-cells, dermal fibroblasts, muscle cells and keratinocytes. Transcriptional re-direction of circuits relevant to tissue repair, such as CD169+ macrophage differentiation, previously shown to drive differentiation of CD4+ Tcells into iTregs, can be increased by SCDIs with an RARα/β ligand or, to a lesser extent, by an RARγ agonist. IL-19, IL-22 and IL-24 production by HaCaT keratinocytes can be amplified by an RARγ agonist or, to a lesser extent, by a partial PPARγ ligand. FGF7/KGF-1, TGFβ and COL1A1 production by dermal fibroblasts can be modestly amplified by an RARγ agonist or, to a more significant extent, by a partial PPARγ ligand. These are key components of sequential action by paracrine circuitry in wound healing. Furthermore, the strength of closure in the healed tissue is greatly dependent on myofibroblast differentiation from fibroblasts. Myoblast (and myofibroblast) differentiation is stimulated by a PPARα ligand. Thus, it would appear that enhancing the actions of RARα/β ligand, RARγ ligand, PPARγ ligand and a PPARα ligand in order, at timely intervals would enhance the healing process. Sequencing of transcriptional effects in participating cell types by administering SCDIs in a temporal sequence either via multiple application or through sequential release times achieved via formulation offer the possibility of choreographing the transcriptional events in skin cells to serve the appropriate underlying requirements of the biological processes in which those cells are engaged. For additional details on these types of revelatory experiments, please see Mascarenhas et al, 2025. That paper represents the first disclosure of the precise transcriptional requirements for enhancing the successful ordering of desired events in this context, based on stimulation cytokine production, known paracrine effects between cell types, and cell surface markers. Thus, the current example teaches the sequential use of SCDIs providing stimulation of RARα/β followed by RARγ, PPARγ and PPARα, in that order. However, because there is overlap between the biological effects triggered by some of these transcriptional stimulators, other potentially useful sequences could include, for example RARα/β followed by PPARγ; or RARγ followed by PPARγ; or PPARγ followed by PPARα. The particulars of the dysfunction being addressed may dictate which actors are most desirable in each circumstance, and in what sequence. It is therefore useful to describe the method by reciting a list of useful modulators and then teach a stepwise application of at least two specified agents from this list that can mimic the way nature uses ligands to modulate a desired biological process in skin tissue at a particular time. Those versed in the field will understand that the methods taught herein enable the possibility of addressing more than one transcriptional mechanism by covalent addition of more than one nuclear receptor ligand to a single peptide. Methods have been disclosed wherein such ligands can be added to both the amino- and carboxy-terminus of a peptide. Examples of such multivalent SCDIs can be found in Mascarenhas et al, 2025.

One way to define the orderly multi-staging of a biological process within skin tissue that is being aided by the methods and compositions of this invention, the temporal sequencing of peptide administration/circulatory levels, and the corresponding temporal sequence of relevant mRNA synthesis and/or protein expression in skin tissue samples is described. The peak level of an administered SCDI peptide (Cmax) can be measured in circulation from blood, plasma or serum samples using, for example, fluorescent tagging of peptides. The peak level of mRNA transcripts in skin tissue extracts can be measured by RNASEQ and other methods known in the art. The peak level of a particular protein in skin tissue extracts can be measured by ELISA and other assays known in the art. Examples can be found in Mascarenhas et al, 2025 and Mascarenhas, 2022. The invention disclosed here specifies at least a four-hour spacing between peaks of first and second SCDI peptide and the same interval between peak levels of Tier A and Tier B protein in skin tissue extracts. It is logical that the “first peptide” specifies transcripts/proteins recited in “Tier A”; whereas “second peptide” specifies transcripts/proteins recited in “Tier B”. Using these very precise parameters and analytes, the invention is strictly defined and enabled.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Sequence Listing in ST.26 XML Format

Incorporation into this description by reference of the uploaded sequence listing file in ST.26 XML format:
Name of XML file: Mascarenhas_Sequence_File_072825.xml
Date of Creation: Jul. 28, 2025
Size of XML file in bytes: 2805

What is claimed is:

1-13. (canceled)

14. A method of transcriptional choreography for selectively modulating a multi-stage biological process by contemporaneously administering to the skin or surface lymphatic tissue of a mammal a first and a second synthetic side-chain-derivatized immodulin peptide 20-60 amino acids long and comprising an amino acid sequence corresponding to either SEQ ID NO:1 or SEQ ID NO:2 covalently attached to a ligand for a nuclear receptor, wherein:
(a) the ligand covalently attached to first peptide is different from the ligand covalently attached to second peptide; and
(b) first peptide is formulated and administered so the peak concentration of first peptide in tissue is achieved at least four hours prior to when peak concentration of second peptide is achieved in the same tissue; and
(c) peak accumulation of CD169 protein in a tissue occurs prior to peak accumulation in the same tissue of a protein selected from a group consisting of IL-19, IL-22, IL-24, and FGF7 and COL1A1;
thereby selectively modulating said multi-stage biological process.

15. The method according to claim 14 wherein first or second synthetic side-chain-derivatized immodulin peptide is covalently attached to a ligand for a nuclear receptor selected from a group consisting of RARα/β, RARγ, PPARα, PPARγ, RXRα and RXRγ.

16. The method according to claim 14 wherein first synthetic side-chain-derivatized immodulin peptide is covalently attached to a ligand for RARα/β nuclear receptor and second synthetic side-chain-derivatized immodulin peptide is covalently attached to a ligand selected from a group consisting of RARγ, PPARα, PPARγ, RXRα and RXRγ.

17. The method according to claim 14 wherein first synthetic side-chain-derivatized immodulin peptide is covalently attached to a ligand for RARγ nuclear receptor and second synthetic side-chain-derivatized immodulin peptide is covalently attached to a ligand selected from a group consisting of PPARα, PPARγ, RXRα and RXRγ.

18. The method according to claim 14 wherein at least two hours after peptide administration, the concentration of administered peptide in the skin or surface lymphatic tissue of a limb distant from the limb where the peptide was administered is at least three-fold higher than the concentration of that same peptide in plasma.

19. The method according to claim 14 wherein sequential release of more than one peptide is achieved by a single dose delivered to the skin or surface lymphatic tissue of a living mammal.

20. The method according to claim 14 wherein the peptide is formulated with purified recombinant human transferrin at a peptide-to-transferrin molar ratio of between 0.01 and 50.

21. The method according to claim 14 wherein the peptide is formulated in a suitable pharmaceutical diluent containing at least 0.001% high-molecular-weight hyaluronic acid.

22. The method according to claim 14 wherein the peptide is formulated with a biodegradable polymer such as poly-lactic-co-glycolic acid or polycaprolactone.

23. The method according to claim 14 used for cosmetic treatment of skin or surface lymphatic tissue in a subject with cosmetic perturbation.
